(19) United States
(12) Patent Application Publication
(10) Pub. No.: US 2011/0208305 A1
(43) Pub. Date: Aug. 25, 2011

(54) COMPOSITE BONE GRAFTS, PARTICULATE BONE-CALCIUM SULFATE CONSTRUCTS, AND METHODS OF TREATING JOINT INJURIES

(75) Inventors: Theodore Malinin, Key Biscayne, FL (US); Temple H. Thomas, Miami, FL (US)

(73) Assignee: UNIVERSITY OF MIAMI, MIAMI, FL (US)

(21) Appl. No.: 13/057,918

(22) PCT Filed: Aug. 5, 2009
(86) PCT No.: PCT/US09/52840

§ 371 Date: Apr. 15, 2011

Related U.S. Application Data
(60) Provisional application No. 61/136,006, filed on Aug. 5, 2008.

(51) Int. Cl. A61F 2/08; A61F 2/28

(52) U.S. Cl. 623/13.14; 623/16.11

(57) ABSTRACT

A solid implantable bone construct (12) shaped like a cylinder, a cone, or a frustum, for anchoring ligament implants. The bone construct (12) can include a bone component and a biocompatible solid component. The bone component can include particulate bone of between 75 and 600 microns, powdered bone of 75 microns or smaller in size, or both. The biocompatible solid component can include calcium sulfate hemihydrate, a calcium phosphate product, or both. The bone component can be between 5 and 50 wt-% of the construct (12) and the biocompatible solid component can be at least 50 wt-% of the construct (12). Also disclosed is a composite graft (10) comprising a first bone dowel (12) and a ligament (20) and a method of securing one bone to another using the composite graft (10).

### FIELD OF THE INVENTION

The present invention is generally directed toward composite bone grafts, surgical implant assemblies comprising the composite bone grafts, and methods of using the same.

### BACKGROUND OF THE INVENTION

Damaged and ruptured cruciate ligaments of the knee (anterior and posterior) can be corrected with surgical treatment. If left untreated chronic pain, instability, laxity and degenerative joint changes are the result. The anterior cruciate ligament (“ACL”) and the posterior cruciate ligament (“PCL”) are frequently subject to traumatic injury, frequently related to sports activities. Because of the mode of inflicted trauma these injuries occur, most frequently, in younger people.

Ligament reconstruction, but not repair, results in the alleviation of pain, reduction in the knee effusion, improved stability and return to normal physical activity. The method of surgical intervention typically employed has been the replacement of the torn ligament with patella tendon of the patient attached to pieces of bone from the tibia and the patella. These are placed in tunnels drilled in the tibia and the femur. The procedure is an effective one, but it is associated with a relatively high morbidity rate and increased operation duration to harvest and prepare autograft. In addition, in case of failure, new autografts are no longer available. For these reasons, allografts and xenografts have been used in lieu of autografts. Xenografts have not met with much success, but allografts provide a number of anatomic structures, which can be employed as ACL and PCL substitutes. Since partial and complete tears of the ACL'S are very common the demand for ACL substitutes allografts is great. It is estimated that in the US over 100,000 ACL and PCL reconstructions are performed annually.

An allograft which anatomically matches the successfully used autografts is the bone-patellar tendon-bone construct. However, the availability of these allografts is limited and hence, many other structures have been used to replace damaged ACL'S and PCL's. These include Achilles tendons, tibialis anterior and tibialis posterior tendons, tendons of hamstring muscles and others. Constructs of fascia lata have also been used in very limited numbers. All of the above mentioned allograft tissues share one problem. They lack either cortical or cancellous bone blocks to which their ends are attached. Therefore, various methods have been devised for attachment of these grafts, under proper tension, in the tibial and femoral tunnels. For these purposes a number of cortical and cancellous bone blocks have been used.

U.S. Pat. No. 7,201,773 issued Apr. 10, 2007 discloses a bone block, a bone-tendon-bone assembly and a method of tendon reconstruction in which at least one tendon replacement is extended between two bone blocks and fixed within each of two bone tunnels in the bones of a joint using interference screws. Thus the patent depends entirely on fixation of the cylindrical bone blocks with interference screws and placement of the ends of tendon grafts into external grooves made in the bone blocks. Dependence on a single screw in each of the canals is a biomechanical weak point. Other associated patents include similar disclosures.

U.S. Pat. No. 6,264,694 issued Jul. 24, 2001 describes a spherical block with a trough going bore and parallel recessed surfaces which enables it to be tied to the end of the ligament graft with a graft secured within a bone tunnel with an interference screw.

U.S. Pat. No. 5,632,748 issued May 27, 1997 describes an anchoring device for bone-patella tendon-bone constructs. The device can be made of bone, metal or other material. The body is tapered and contains a groove to receive a fixation screw and two curved recesses to hold a tendon which is looped over the device. U.S. Pat. No. 5,562,669 issued Oct. 18, 1996 discloses a bone-patella tendon-bone anchor device made of autologous bone plugs made from the cores of tunnels cored in the patient. The plugs can be also made from allograft bone. The cylindrical plug is provided with two parallel grooves which provide recesses for seating the tendon.

U.S. Pat. No. 4,755,593 issued July 1988 relates to xenografts. Xenograft tissues are highly antigenic. Therefore attempts are made to reduce antigenicity prior to transplantation into humans. The patent describes the tanning or cross-linking technique to achieve this. Gluteraldehyde is used to this end. This patent as well as U.S. Pat. No. 4,400,333, U.S. Pat. No. 4,755,593 and PCT Publication No. WO 84/03036 do not specifically deal with ACL/PCL replacement xenografts.

### SUMMARY OF THE INVENTION

The present invention in various embodiments is directed to a fascia lata composite graft for use in cruciate ligament reconstruction. Composite grafts as described in this invention can be also composed of tendons and fibrous tissues other than fascia lata. The present invention applies to the reconstruction of the cruciate ligaments of the knee. In the inventive surgical installation conical tunnels are drilled in the tibia and the femur with narrower portions of the same directed towards the knee joint. Conical allograft blocks prepared from particulate bone mixed with calcium sulfate hemihydrate (CaSO₄.½H₂O), calcium phosphate, or other biocompatible solid materials match the conical tunnel and retain the tendon grafts. Tendon replacement grafts placed through these constructs produce constant tension and help to impact the retaining blocks. Fixation with interference screws or any other screws, pins, nails or similar entities is unnecessary and is eliminated.

The tendon replacement grafts are extended between the bone conical cylinders through the central bone of each construct. The tendon replacement graft is first passed through the tibial block which is then inserted into the tibial tunnel by being pulled into the tibial tunnel by the replacement graft and the attached sutures. The graft is passed through the femoral tunnel and through the femoral conical allograft block. To secure the tendon in the tibial block a knot can be tied therein, or the tendon is folded on itself and sutured together. Alternatively a crimp can be applied to the tendon preventing it from sliding through the block. Once the ligament passes through the femoral allograft block, a desired tension is applied to the graft. The block is press fitted into the femoral tunnel and the graft secured in place an identical matter to that described for the tibial block.

Calcium sulfate hemihydrate, when mixed with water creates a durable and hard construct. A cylinder made of calcium sulfate measuring 20 mm in length and 10 mm in diameter will resist a compressive force of 1200-1400 N. Experiments in non-human primates have shown that calcium sulfate cylinders become resorbed by six weeks, which is before they are replaced by new bone. However, it has unexpectedly been learned that plugs of calcium sulfate mixed in adequate proportions with microparticulate bone become replaced with regenerated host bone in 6 weeks. Thus, a beneficial aspect of the devices disclosed herein is that the bone dowels including a bone component and a biocompatible solid provide improved healing and attachment of the replacement ligament over time.

The present invention overcomes the current problems with shortages of ACL/PCL substitute grafts by making it possible to use fascia lata allografts and composite bone dowels using particulate or powdered bone materials.

An object of the invention is also to provide a conical bone allograft which allows for implantation and retention under desired tension of fascia lata and other tendon allografts.

Another object of the invention is to utilize a press-fit conical bone-comprising blocks for the retention of ACL/PCL substitute grafts. This eliminates the need for the interference screws or other fixation devices.

It is also an object of the invention to provide pre-shaped bone derived structures that will effectively promote new bone growth and accelerate healing. This is achieved using openings connecting to the central bone which can be left open to allow for the in-growth of tissue from the patient or can be fitted with autologous, non-demineralized microparticulate bone, demineralized bone particles or other growth promoting substances.

It is an additional object of the invention to construct blocks, cylinders cones and other configurations of mixtures of calcium sulfate hemihydrate and microparticulate or other particulate bone.

It is also an additional object of the invention to construct calcium sulfate hemihydrate-tendon calcium sulfate construct of inventive design to provide an anatomically suitable cruciate ligament replacements.

It is also an object of the invention to create ACL/PCL substitute assemblies which can be easily handled by surgeons eliminating the need for shaping allografts during surgery.

### BRIEF DESCRIPTION OF DRAWINGS

A fuller understanding of the present invention and the features and benefits thereof will be obtained upon review of the following detailed description together with the accompanying drawings, in which:

FIG. 1 shows a view of an exemplary fully-assembled composite graft including two bone dowels and a ligament.

FIGS. 2a and 2b show cross sectional views of the bone dowels in FIG. 1 taken along cut lines 2a-2a and 2b-2b, respectively.

FIG. 3 shows an exemplary bone dowel according to the invention from FIG. 3a. a side view, FIG. 3b. a front view and FIG. 3c. a back view.

FIG. 4 depicts a 3 cm wide strip of fascia lata fashioned into a tubular structure by whip-stitching technique. Biochemically the strength of the graft of the invention is equal to or exceeds that of anterior cruciate ligaments.

FIG. 5 depicts photographs of fascia lata allograft inserted into conical bone constructs designed to retain the graft in the tibial and femoral tunnels under tension.

FIG. 6 depicts a perspective views the inventive fascia lata construct inserted into a cylinder made of microparticulate bone and CaSO₄.½H₂O.

FIG. 7 depicts a photograph of a cylinder, with a central perforation made of calcium sulfate hemihydrate. The construct will withstand compressive force of 1500 before mechanical failure occurs.

FIG. 8 depicts a cylinder with a central perforation made of 50% bone particles mixed with calcium sulfate hemihydrate. The cylinder will withstand compressive load between 1200 and 1500 N.

FIG. 9 depicts an X-ray of cylinders made of a mixture of cortical bone particles, 50% by weight, and calcium sulfate hemihydrate. Darker areas within the cylinders are clumps of particulate bone.

FIG. 10 depicts X-rays of distal femur with a healed defect (arrow) and a control defect. The healed defect was filled with calcium sulfate hemihydrates (CaSO₄.½H₂O)-particulate bone mixture.

FIG. 11 depicts gross specimen of a distal femur of an animal with a defect filled with calcium sulfate hemihydrate (CaSO₄.½H₂O) bone particle mixture. Six weeks after installation, the mixture is being replaced with new bone from the host.

### DETAILED DESCRIPTION OF INVENTION

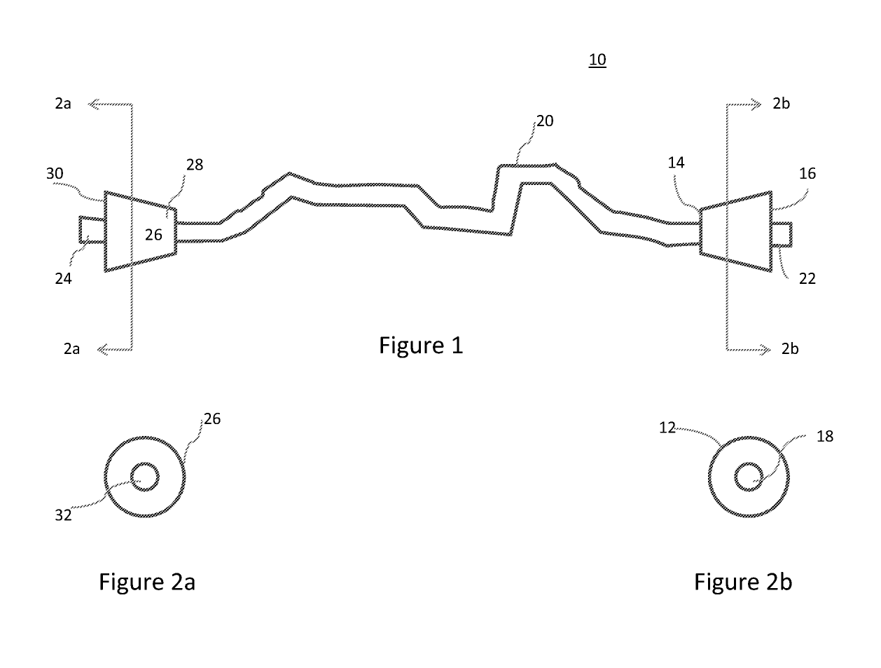

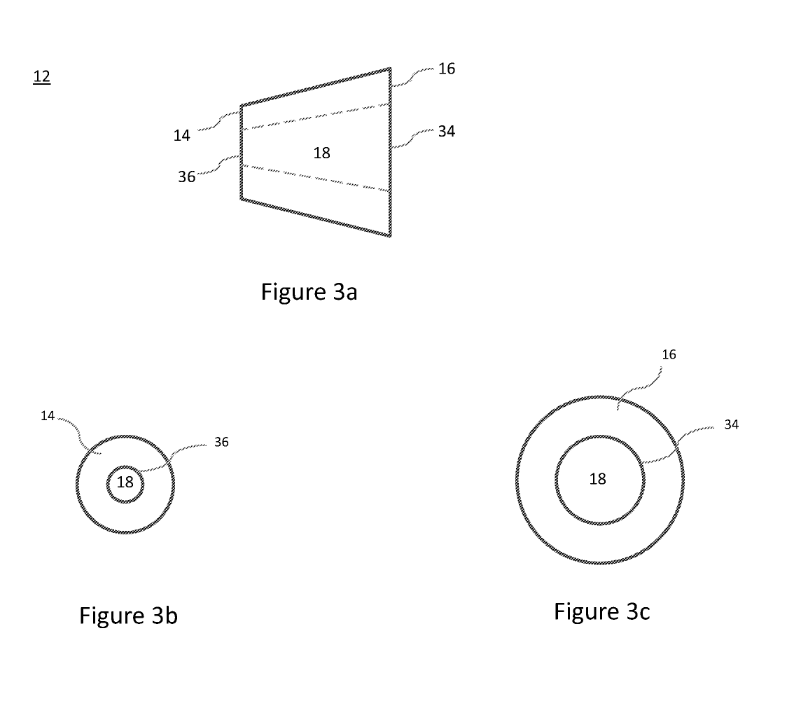

As shown in FIGS. 1-3, one embodiment of the invention is directed to a composite graft (10) comprising a first bone dowel (12) which has the shape of a frustum, having a first proximal end (14) and a first distal end (16), the first bone dowel (12) containing a first axial bore (18) extending from the first proximal end (14) to the first distal end (16), wherein the first distal end (16) has an area greater than the area of the first proximal end (14). The graft (10) also includes a ligament (20) with a first end (22) and a second end (24), wherein the first end (22) is attached to the first bone dowel (12) within the first axial bore (18) such that the first proximal end (14) of the first bone dowel (12) is closer to the second end (24) of the ligament (20) than is the first distal end (16).

As used herein, “frustum” is used to refer to the part of a solid, such as a cone or pyramid, between two usually parallel cutting planes. In addition to cone frusta, additional examples include pentagonal, square, triangular, hexagonal, heptagonal and octagonal frusta. The frustum disclosed herein can be either right frusta or oblique frusta.

The composite graft (10) can further comprise a second bone dowel (26) the shape of a frustum, having a second proximal end (28) and a second distal end (30), the second bone dowel (26) containing a second axial bore (32) extending from the second proximal end (28) to the second distal end (30), wherein the second distal end (30) has an area greater than the area of the second proximal end (28). The second end (24) of the ligament (20) can be attached to the second bone dowel (26) within the second axial bore (32) such that the proximal ends (14, 28) of the first and second bone dowel (12, 26) are closer to each other than the distal ends (16, 30) of the first and second bone dowel (12, 26) are to each other.

In the composite graft (10), the first axial bore (18) and the second axial bore (32) have a distal opening (34) and a proximal opening (36). The distal opening (34) and the proximal opening (36) of each axial bore (18, 32) can have the same area and shape, e.g., can be cylindrical. Alternatively, the distal opening (34) can have a larger area than the proximal opening (36) for one or both axial bores (18, 32). Further, the first or second bone dowel (12, 26) of the composite graph can comprise a plurality of small holes.

The ligament (20) of the composite graph (10) can comprise a soft tissue selected from the group consisting of fascia lata, another fascia, pericardium, dura mater, tendons, skin and any of the skin components and a combination thereof. The tendon can be selected from the group consisting of tibialis anterior tendon, tibialis, posterior tendon, patellar tendon, quadriceps tendon, adductor magnus tendon, peroneus tendon, Achilles' tendon, gracilis tendon, and a combination thereof.

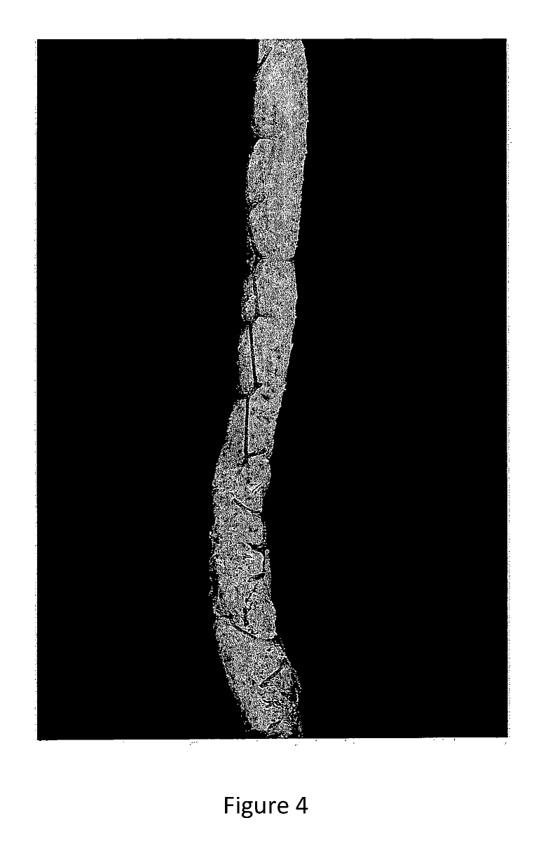

The fascia lata can comprise fascia lata fluted, folded or rolled and whip-stitched into a desired diameter and configuration. FIG. 4 shows a picture of a 3 cm wide strip of fascia lata fashioned into a tabular structure by whip-stitching technique. Biochemically the strength of the graft of the invention is equal to or exceeds that of anterior cruciate ligaments.

In a preferred embodiment, the composite graft (10) is a ligament replacement for a patient's tendon. The tendon being replaced can be selected from the group consisting of tibialis anterior tendon, posterior tendons, patellar tendons, quadriceps tendon, adductor magnus tendon, peroneus tendon, Achilles' tendon, patellar tendon, semitendonosus tendon and a combination thereof.

The ligament (20) of the composite graph (10) can comprise at least a part of a natural ligament selected from the group consisting of pericardium, dura mater, fascia, skin and any of its components, and a combination thereof. The fascia can be a fascia covering a muscle. For example, the fascia can be selected from the group consisting of abdominal fascia, deltoid fascia, transversalis fascia, scarpas's fascia, pectoral fascia, fascia iliaca, tibialis fascia, an lumbo-dorsal fascia and a combination thereof.

The composite graft (10) can have first and second bone dowel (12, 26) comprising bone particles selected from the group consisting of cancellous bone, cortical bone, cortico-cancellous bone and a combination thereof. Further, the first and second bone dowel (12, 26) can comprise calcium sulfate hemihydrates, a calcium phosphate product, or both. Additionally, the first and second bone dowel (12, 26) can comprise decalcified microparticulate bone particles, demineralized bone particles, calcium sulfate hemihydrate, a calcium phosphate product, or a combination thereof.

The composite graft (10) of the invention can be used for implantation in a patient. In such a method/use, the first and second bone dowel (12, 26) can be autograft cortical bone from the patient, or selected from the group consisting of allograft cancellous bone, xenograft cancellous bone, allograft cortical bone, xenograft cortical bone, bioabsorbable synthetic material, ceramic, and a combination thereof.

In another aspect, the composite graft (10) can have the ligament (20) attached at the proximal ends (14, 28) to the first and second bone dowels (12, 26) by a knot, a suturing ligament, a doubled ligament, a folded over ligament, a crimp, or a combination thereof.

The composite graft (10) of the invention can be made without a metal part. This provides a significant advantage over grafts with metal parts, because the interaction of the metal and the body would not be a concern if the graft has no metal.

The composite grafts (10) of the invention may be used to secure bone connection in a patient between a first bone and a second bone. The method can comprise the steps of: providing a composite graft (10) to replace a damaged ligament in the patient, wherein the composite graft (10) comprises, a first bone dowel (12) having the shape of a frustum and having a first proximal end (14) and a first distal end (16). The first bone dowel (12) containing a first axial bore (18) extending from the first proximal end (14) to the first distal end (16), where the first distal end (16) has an area greater than the area of the first proximal end (16). The composite graft can also include a ligament (20) with a first end (22) and a second end (24), wherein the first end (22) is attached to the first bone dowel (12) within the first axial bore (18) such that the first proximal end (14) of the first bone dowel (12) is closer to the second end (24) of the ligament (20) than is the first distal end (16). Additional steps include drilling a first tunnel through a first bone of the patient, drilling a second tunnel through a second bone of the patient, and threading the second end (24) of the ligament (20) through the first tunnel and second tunnel in any order. A second bone dowel (26) can be provided having substantially the same features as the first bone dowel (12). The second end (24) of the ligament (20) can be threaded through the second proximal end (28) of the second bone dowel (26) and attached within the second axial bore (32) such that the proximal ends (14, 28) of the first and second bone dowel (12, 26) are closer to each other than the distal ends (16, 30) of the first and second bone dowel (12, 26) are to each other. This method securing the first bone to the second bone and producing a secured bone connection.

Ligament repair techniques are well known in the art. An exemplary technique is disclosed by Stephen M. Howell, et al., “Compaction of a Bone Dowel in the Tibial Tunnel Improves the Fixation Stiffness of a Soft Tissue Anterior Cruciate Ligament Graft: An In Vitro Study in Calf Tibia,” America Journal of Sports Medicine, Vol. 33, 719-725 (2005).

In this method, the first distal end (16) of the first bone dowel (12) and the second distal end (30) of the second bone dowel (26) can have a area greater than or equal to the area of the first or second tunnel. Further, the first tunnel, the second tunnel or both, can comprise a wide end and a narrow end, the wide end adapted for receiving and securing the first or second bone dowel and to prevent the first or second bone dowel from traversing the first or second tunnel.

The methods of the invention may be used for repairing a knee of a patient, where the damaged ligament in the patient is an anterior cruciate ligament, the first bone is a tibia, and the second bone is a femur. The method of the invention provides a significant advantage in that the secured bone joint which is formed does not contain any metal or bone screws.

The method of the invention may be used to repair a bone joint by creating a plurality of secured bone connections between a first bone and a second bone. For example, the bone joint can be a knee and the plurality of secured bone connections can be two secured bone connections. In other words, the methods of the invention may be used to treat or stabilize damaged and ruptured cruciate ligaments of the knee (anterior and posterior) including treatment of torn anterior cruciate ligament and the posterior cruciate ligament.

Another aspect of the invention is directed to a solid implantable bone construct (12) comprising calcium sulfate hemihydrates, a calcium phosphate product, or both, and (a) particulate bone of between 75 and 600 microns, (b) powdered bone of 75 microns or smaller in size, or (c) both. The solid implantable bone construct (12) may be in the shape of a cylinder or a frustum. The cylinder or frustum can include an axial bore.

The particulate bone of the solid implantable bone can be freeze dried, frozen bone or unfrozen bone. The particulate bone can be a mixture of sizes between 75 and 600 microns. The calcium phosphate product can be selected from the group consisting of calcium deficient apatite, hydroxyapatite, beta-tricalcium phosphate, biphasic calcium phosphate, and a combination thereof.

The bone component can be 5 to 50 wt-% of the bone dowels (12, 26) disclosed herein, or 5 to 30 wt-%, or 7.5 to 30 wt-% or 10 to 25 wt-%, or 10 to 20 wt-%. The biocompatible solid component can be at least 50 wt-% of the bone dowels (12, 26) disclosed herein, or at least 70 wt-% or at least 75 wt-%, or at least 80 wt-%.

The inventors have discovered that when calcium sulfate is used to fill a bone defect it dissolves at a rapid rate of approximately 1 mm per day from the exterior to the center. This resorption causes precipitation of calcium phosphate deposits which stimulates formation of new bone. However, the process is not rapid enough to fill the void with new bone. Therefore, after calcium sulfate is resorbed the void remains. This is overcome by the combination of particulate bone and calcium sulfate hemihydrates as disclosed herein. This unexpected discovery facilitates bone growth and regeneration and provides for a method and source for producing various constructs for bone repair. Devices formed using such mixtures can be in the shape of dowels, rectangles, spheres, tubes and other constructs adapted for a variety of applications. The mixture can also be provided in a dry form or, shortly after water is added, as a putty-like material. In addition, constructs to match specific anatomic defects and locations can be prepared, including spinal fusions.

Of all calcium sulfate formulations, it has been determined that only calcium sulfate hemihydrates have the ability to form cement-like composition when mixed with water. Such material in pure form is usually resorbed by human bone from two to seven weeks. This material is resorbed faster than it can be replaced by new bone. It has been determined that addition of bone particles to calcium sulfate hemihydrates accelerates bone replacement and allows for the retention of the composition in the bone void, until such time as it is replaced with new bone.

Based on these discoveries, one embodiment of the invention disclosed herein is a dry mixture or putty that can be used for filing voids within or between bones. In one embodiment, the bone void fling composition can include (a) a bone component comprising bone particulate of between 75 and 600 microns, powdered bone of 75 microns or smaller in size, or both, wherein said solid implantable bone construct is a shape selected from a cylinder, a cone, or a frustum; and (b) a biocompatible solid component comprising calcium sulfate hemihydrate, a calcium phosphate product, or both. The bone component comprises between 5 and 50 wt-% of the bone filing composition and the biocompatible solid component comprises at least 50 wt-% of the bone filing composition based on the total amount of bone component and biocompatible solid component. Any combinations of bone component and biocompatible solid component disclosed herein can also be used.

The bone filing composition can be distributed as a dry mixture for use filing voids within bones or between bones. Prior to introducing the bone filing composition, water can be added to the dry mixture. Once water is introduced, the water-bone component-biocompatible solid component mixture has a putty-like, viscous consistency and can be applied in bone voids. The water triggers an exothermic reaction with the biocompatible solid component and the mixture begins to cure and solidify. This technique can be used to fill bone voids within a bone or between bones. An exemplary procedure includes spinal fusion, where the bone filing composition is applied between vertebras.

The bone void filing composition can include water or be a substantially dry mixture. The bone void filing composition can include water in an amount ranging from 0.10 ml and 0.35 ml per gram of mixture of said bone component and said biocompatible solid component, or ranging from 0.15 ml/gm to 0.32 ml/gm, or ranging from 0.2 ml/gm to 0.30 ml/gm.

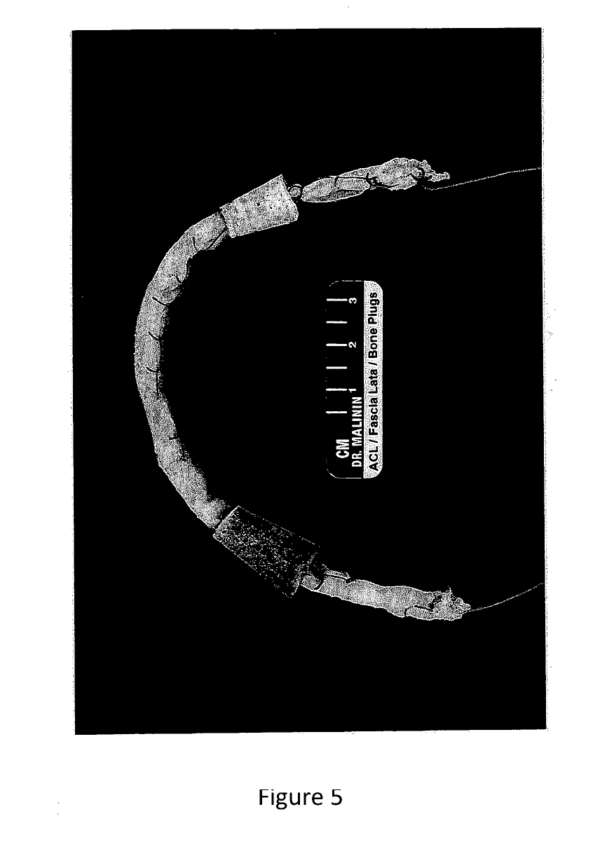

Complete graft composites are shown in FIGS. 5 & 6. A complete fascia lata ligament with bone cone constructs is illustrated in FIG. 5. In accordance with the present invention, the ACL or PCL replacement allograft can be inserted surgically through an arthroscopic procedure an open arthrotomy. The description of the invention is primarily directed toward knee reconstruction.

A number of surgical procedures and modifications of the same are used in cruciate ligament reconstructions. Techniques are fully explained in the books “Crucial Ligaments’ John A. Feagin, Jr. ed, 1994 and Campbell's Operative Orthopaedics, 1998, chapter 29. These are incorporated herein by reference. In the standard ACL reconstruction, the knee is prepared by drilling the femoral tunnel through the intercondylar notch medially. The tibial tunnel is drilled starting between the tibial tubercle and the medial edge of the proximal tibia. The tibial tunnel terminates at the site of the medial attachment of the ACL. The tunnels are drilled using a cannulated reamer 8 to 12 in diameter. The grafts are pulled through these tunnels which are placed anatomically, i.e. approximating the normal direction of the ACL.

After the tunnels have been made the allograft assembly with pre-shaped bone blocks to which the ACL replacement grafts have been attached are pulled through the tunnels, usually by surgical sutures inserted in the grafts. The bone blocks are then secured in the tunnels by interference screws or other fixation devices. If a soft tissue allograft such as a tibialis anterior tendon is not attached to bone it can be secured in place by sutures tied to metal posts or by screws which transfix the graft. In the latter case the fixation is not or as strong as it is with host bone blocks and in interference screws.

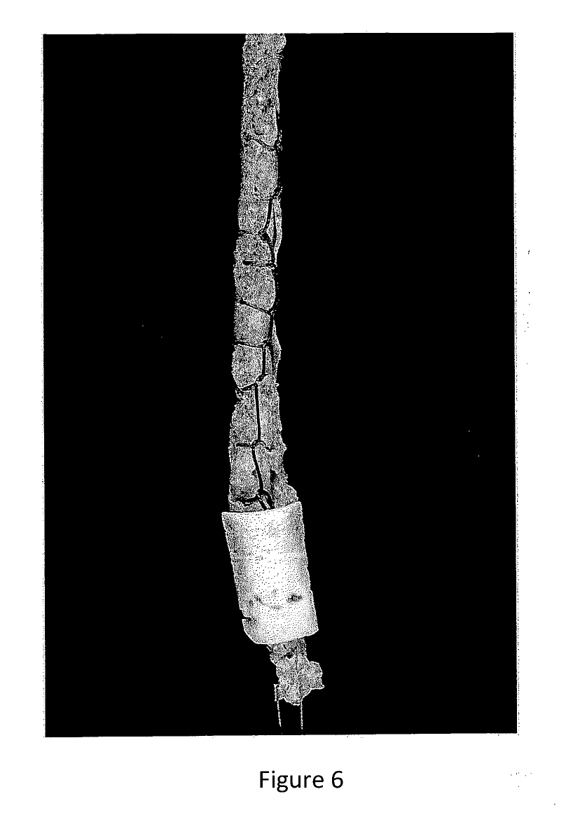

The present invention describes a technique which is substantially different from the existing techniques of ACL replacement allograft insertion and fixation. As seen in FIG. 6 the graft prepared from fascia lata is passed through the central bore of a conically shaped bone dowel construct. First, the replacement tendon is passed through the tibial cone with the wide portion of the cone being on the outside. The replacement tendon is then either knotted, folded on itself and sutured or is retained with a crimp. This prevents the replacement tendon from slipping through the central bore of the cylinders.

A conical tibial tunnel is drilled to correspond in shape and dimensions to the tibial conical cylinders. The graft to which sutures have been attached is then passed through the tibial tunnel and the conical construct is pulled and press fitted into the tunnel. The graft is then pulled through the femoral tunnel and through the conical construct with the narrow portion directed towards the knee joint. The graft is then pulled to a desired tension. The bone or CaS0₄ conical cylinder press fitted into conically drilled femoral tunnel. The tendon is secured by either tying a knot, holding on itself and suturing or by applying a crimp.

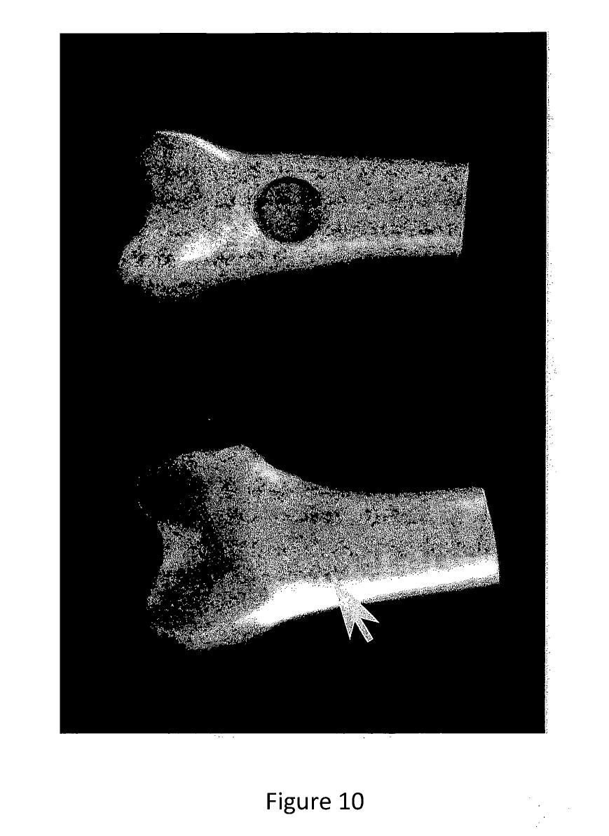

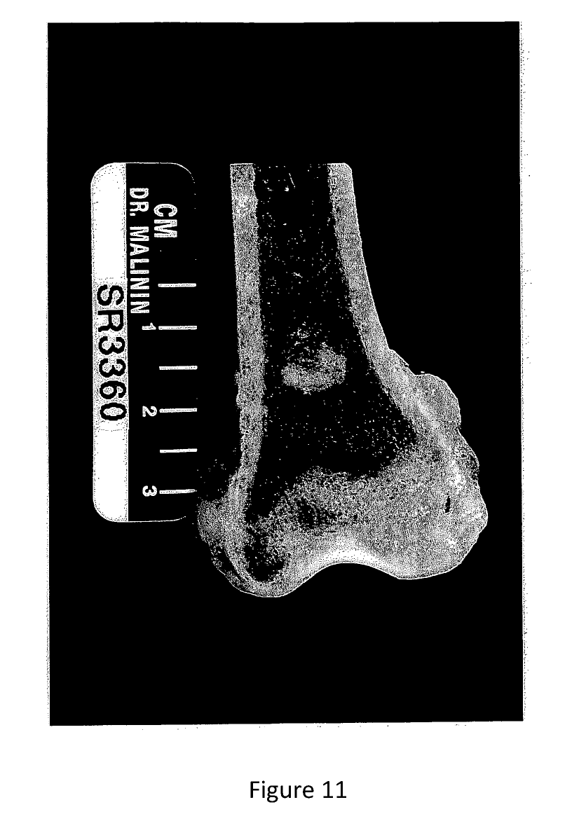

FIG. 10 shows X-rays of distal femur with a healed defect (arrow) and a control defect. The healed defect was filled with calcium sulfate hemihydrate-particulate bone mixture. In addition, FIG. 11 depicts Gross specimen of a distal femur of an animal with a defect filled with calcium sulfate hemihydrates-bone particle mixture. Six weeks after instillation the mixture is being replaced with new bone from the host.

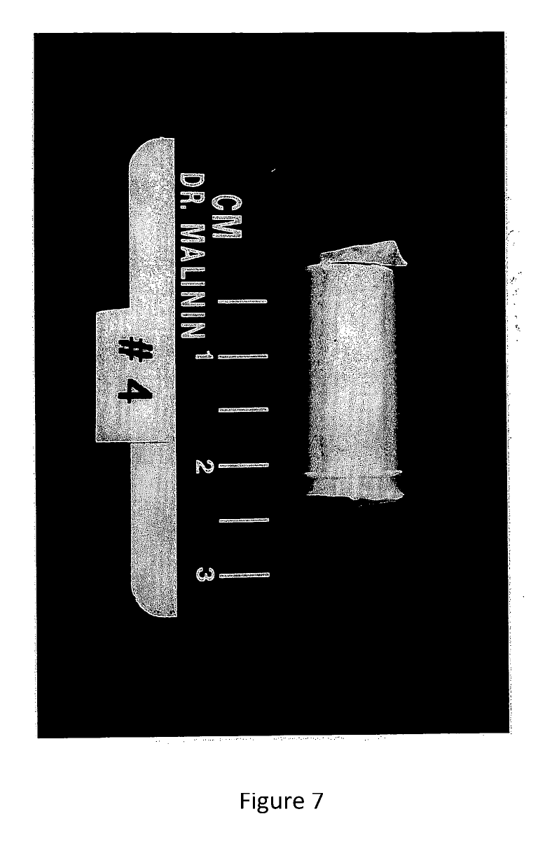

Another alternative to using conically shaped blocks is to use a conventional cylindrical block of bone or CaSO₄.½H₂O bone particle components as shown in FIGS. 7 and 9. If straight cylinders are used for the retention of the graft these are secured in the patient's tibial and femoral tunnels by interference screws or other fixation devices.

While the technology herein has been described in connection with exemplary illustrative non-limiting implementations, the invention is not to be limited by the disclosure. The invention is intended to be defined by the claims and to cover all corresponding and equivalent arrangements whether or not specifically disclosed herein. All patents, patent applications and references cited in this disclosure are incorporated by reference in their entirety.

### EXAMPLES

### Example 1

### Testing the Biochemical Properties of Constructed Composite Grafts

Fascia lata tubes are strong biomechanical constructs. Their biomechanical properties as compared to other ACL replacement allografts are given in table 1.

TABLE 1
Comparative Strength of ACL Replacement ligaments.

| Ligament | No. tested | Load to failure N (mean) |
|---|---|---|
| Tibialis anterior tendon | 19 | 822 |
| Achilles tendon | 8 | 2204 |
| Patellar ligament (whole) | 8 | 2521 |
| Patellar ligament (½) | 10 | 1677 |
| Peroneous longus tendon | 12 | 876 |
| Tibialis posterior tendon | 10 | 900 |
| Gracilis tendon | 10 | 697 |
| ACL | 15 | 867 |
| Fascia lata 3 cm strips | 8 | 994 |

### Example 2

### Preparation of Solid Implantable Bone Constructs

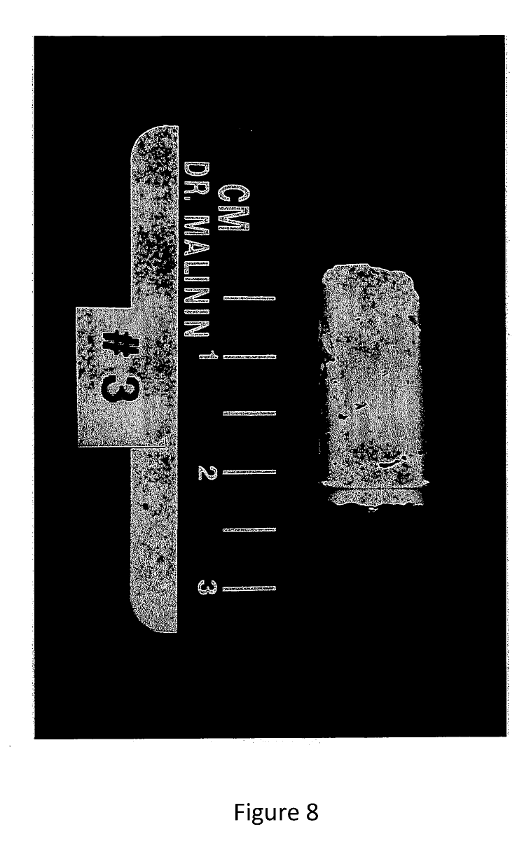

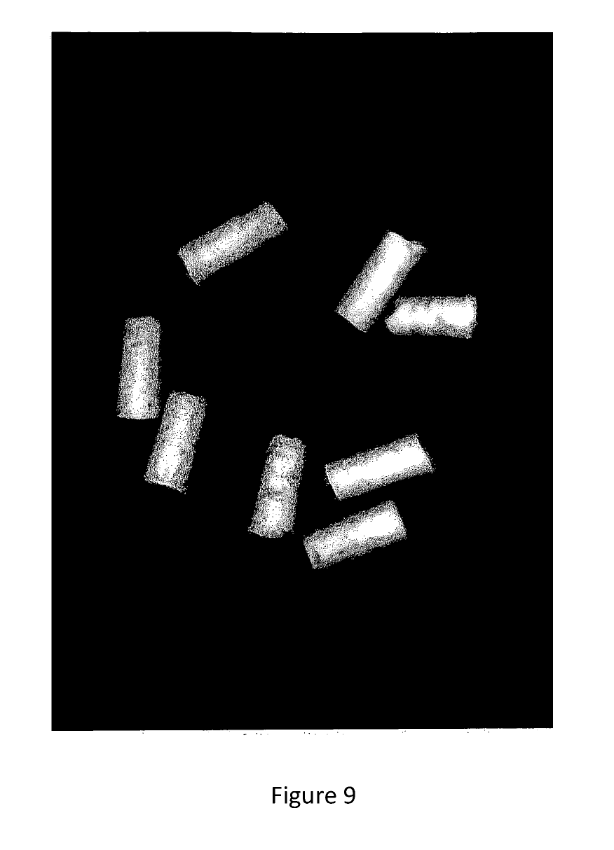

Calcium sulfate studies have been reviewed by Alexander et al (ICRC Critical Reviewers in Biocompatibility, 1987; 4:43). Calcium sulfate is biocompatible, does not evoke inflammatory response, and does not inhibit bone formation. Eventually calcium sulfate may be replaced by new bone, but resorption of calcium sulfate is more rapid than the rate of its replacement with new bone. Clinical studies with calcium sulfate implanted alone or mixed with demineralized bone matrix, autologous bone or bone morphogenic protein (BMP) reveal that calcium sulfate alone is as effective as it is in compilation with the above listed substances (LeGeros RZ et al, Bioactive Bioceramics. In Orthopaedic Biology and Medicine: Musculoskeletal Tissue Regeneration (WS Pietrzack ed) Human Press, 2008 incorporated herein by reference) calcium sulfate degrade within 5-6 weeks. However, it has been demonstrated by the inventors that compact composite calcium sulfate hemihydrate (Plaster of Paris)-bone particle cylinders remain unabsorbed for over six weeks. The present invention is related to mixing calcium sulfate hemihydrate in various proportions with undemineralized microparticulate bone and implanting cylinders made of the above mixture into experimental animals (non-human primates). Unlike mixtures of calcium sulfate with autologous bone, demineralized bone matrix or BMP the cylinders of calcium sulfate hemihydrate mixed with microparticulate bone induce active osteogenesis and are replaced with newly regenerated bone as shown in FIGS. 7 to 11.

Of all varieties of CaS0₄ available only calcium sulfate hemihydrates (Plaster of Plaster)(CaSO₄.½H₂O) is suitable for constructing biomechanically sound cylinders. The amount of water needed to produce material which hardens in 5 minutes is 1.0 ml per 5 gm of calcium sulfate. In this proportion cylinders of calcium sulfate hemihydrate measuring from 10 to 22 m in length and 10 mm in diameter will withstand compressive load of 1200 to 1400 N. This is comparable to bone plugs of the same dimension made from compact bone of human distal femur (1,200-1,50N). Dowels prepared from calcium sulfate hemihydrate with 25% by weight microparticulate bone will withstand compressive loads from 800 to 1400 N. Dowels prepared from calcium sulfate hemihydrates with 50% by weight bone will withstand compressive forces in a similar range 844-1246N. However, it has been determined that if amount of microparticulate bone exceeds 50% the mixture becomes brittle and unsuitable for support of ACL replacement ligament. Therefore the invention encompasses bone particle calcium sulfate hemihydrate (CaSO₄.½H₂O) mixtures from 0-50% by weight.

The procedure for the preparation of bone particle CaS0₄ ½ hydrate is as follows. Bone particles of cortical and cancellous bone measuring from 25-600 microns are prepared according to the previously described method (U.S. Pat. No. 7,335,381, Malinin et al, 2008). The bone particles are mixed with calcium sulfate hemihydrate (CaSO₄.½H₂O) a 50:50% proportion or less. Water is than added to the mixture and the paste put into a mould. It will harden within 5 minutes.

It is noted that the reaction causing solification is triggered when water is added. Thus, the solid mixture of some combination of calcium sulfate hemihydrate, calcium phosphate, and bone should be mixed prior to addition of water. The procedures used for production of compositions with calcium phosphate are identical.

### Example 3

### Compression Testing

Calcium sulfate hemihydrates have solubility in water that is higher than that of calcium sulfate dehydrate or anhydrous calcium sulfate. Therefore when properly mixed with water, calcium sulfate hemihydrates will dissolve and then recrystallize to form gypsum cement. The formation of gypsum cement depends on the amount of water added to calcium hemihydrates. The formation of cement is accompanied by heat generation. The period during which heat is produced can vary from 3 to 5 minutes to 45 minutes.

To produce a paste which hardens in 5 to 10 minutes a mixture of 0.25 ml of water with 1 gm of calcium sulfate hemihydrates can be used. However, the addition of bone microparticles to calcium sulfate hemihydrate changes its characteristics when mixed with water. For example, a mixture of 30 wt-% of calcium sulfate hemihydrates and 70% bone particles will not solidify and will remain a paste. In addition, a paste that hardens in 5-10 minutes is produced when bone microparticles are mixed with calcium sulfate hemihydrate in a 50:50 ratio and 1.1 ml of water is added to 4 gm of the mixture.

It is also noted that, it excess water (0.34 ml per gram for 50:50 bone: calcium sulfate hemihydrates mixtures) is used instead of 0.25 ml the mixture solidifies only partially, and the composition crumbles. Further increase in water (0.37 ml/gm of mixture) prevents hardening of the mixture.

Addition of bone particles to calcium sulfate hemihydrate delays solidification of the mixture. In compositions with 50% bone particles, it may take up to 24 hours to solidify.

| 0% Bone | 50% Bone | 30%% Bone | 20% Bone |
|---|---|---|---|
| 2476 | 842 | 1467 | 2157 |
| 2672 | 1143 | 1293 | |
| 2678 | 1156 | 680 | |
| 2431 | 1246 | 717 | |
| 2769 | 844 | 800 | |
| Avg. = 2368 | Avg. = 1046 | Avg. 991 | |

The biomechanical properties of pure gypsum cement composition and gypsum cement mixed with bone particles differ. This is demonstrated in Table 2, below, which shows the results of compression testing of 10 mm diameter and 2.5 cm length cylinders. The force in Newtons required to produce failure using calcium sulfate hemihydrates with a given amount of bone particulate is as follows:

By comparison identical cylinder prepared from bone resulted in an average of 1668 Newtons

Biomechanically, longitudinal strength of calcium sulfate hemihydrates cylinders exceeds strength of cortico-cancellous bone of the same dimensions. When 20 wt-% of bone particles is added the biomechanical properties of calcium sulfate hemihydrates constructs are retained. However, when 30 or 50% bone particles are mixed with calcium sulfate hemihydrates (CaSO₄.½H₂O) biomechanical strength decreases, but, not dramatically so when compared to comparable bone structures.

It is to be understood that while the invention in has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

What is claimed:

1. A composite graft (10) comprising:
a first bone dowel (12) having the shape of a frustum, said first bone dowel having a first proximal end (14) and a first distal end (16) and a first axial bore (18) extending from the first proximal end (14) to the first distal end (16), wherein said first distal end (16) has an area greater than an area of said first proximal end (14); and
a ligament (20) with a first end (22) and a second end (24), wherein said first end (20) is attached to said first bone dowel (12) within said first axial bore (18) such that the first proximal end (14) of said first bone dowel (12) is closer to the second end (24) of said ligament (20) than the first distal end (16),
wherein said first bone dowel (12) comprises:
(a) a bone component comprising particulate bone of between 75 and 600 microns, powdered bone of 75 microns or smaller in size, or both, and
(b) biocompatible solid comprising a calcium sulfate hemihydrate, a calcium phosphate product, or both,

2. The composite bone graft (10) of claim 1 wherein said bone component comprises between 5 and 50 wt-% of said first bone dowel (12) and said biocompatible solid component comprises at least 50 wt-% of said first bone dowel (12).

3. The composite bone graft (10) of claim 1 wherein said bone component comprises between 5 and 30 wt-% of said first bone dowel (12) and said biocompatible solid component comprises at least 70 wt-% of said first bone dowel (12).

4. The composite graft (10) of claim 1 further comprising:
a second bone dowel (26) which has the shape of a frustum, having a second proximal end (28) and a second distal end (30), the second bone dowel (26) containing a second axial bore (32) extending from the second proximal end (28) to the second distal end (30), wherein said second distal end (30) has an area greater than an area of said second proximal end (28);
wherein said second end (24) of said ligament (20) is attached to said second bone dowel (26) within said second axial bore (32) such that the proximal ends (14, 28) of the first and second bone dowel (12, 26) are closer to each other than the distal ends (16, 24) of the first and second bone dowel (12, 26) are to each other.

5. The composite graft (10) of claim 4 wherein said ligament (20) is attached at the proximal ends (16, 30) to said first and second bone dowels (12, 26) by a knot, a suturing ligament, a doubled ligament, a folded over ligament, a crimp, or a combination thereof.

6. The composite graft (10) of claim 4 wherein the first axial bore (18), the second axial bore (32), or both are of uniform dimensions.

7. The composite graft (10) of claim 4 wherein the first axial bore (18), the second axial bore (32), or both, have a distal opening (34) and a proximal opening (36), wherein an area of said distal opening (36) is greater than an area of said proximal opening (36).

8. The composite graft (10) of claim 1, wherein said ligament (20) comprises a soft tissue selected from the group consisting of fascia lata, another fascia, pericardium, dura mater, tendons, skin and any of the skin components and a combination thereof.

9. The composite graft (10) of claim 8, wherein said fascia lata comprises fascia lata fluted, folded or rolled and whip-stitched into a desired diameter and configuration.

10. The composite graft (10) of claim 1 wherein said composite graft (10) is a ligament replacement for a patient's tendon.

11. The composite graft (10) of claim 10, wherein said patient's tendon is selected from the group consisting of tibialis anterior tendon, posterior tendons, patellar tendons, quadriceps tendon, adductor magnus tendon, peroneus tendon, Achilles' tendon, patellar tendon, semitendonosus tendon and a combination thereof.

12. The composite graft (10) of claim 1 wherein said ligament (20) comprises at least a part of a natural ligament selected from the group consisting of pericardium, dura mater, fascia, skin and any of its components, and a combination thereof.

13. The composite graft (10) of claim 12 wherein said fascia is a fascia covering a muscle.

14. The composite graft (10) of claim 13 wherein said fascia is selected from the group consisting of abdominal fascia, deltoid fascia, transversalis fascia, scarpas's fascia, pectoral fascia, fascia iliaca, tibialis fascia, a lumbo-dorsal fascia and a combination thereof.

15. The composite graft (10) of claim 1 wherein said bone component comprises a bone selected from the group consisting of cancellous bone, cortical bone, cortico-cancellous bone and a combination thereof.

16. The composite graft (10) of claim 1 wherein said bone component comprises decalcified microparticulate bone, demineralized bone, or a combination thereof.

17. The composite graft (10) of claim 1 wherein said composite graft (10) does not comprise metal.

18. A method for providing one secured bone connection in a patient between a first bone and a second bone, the method comprising:
(a) providing a first and second bone dowel (12, 26) having the shape of a frustum, said first and second bone dowels (12, 26) each having a proximal end (14, 28) and a distal end (16, 30) and an axial bore (18, 32) extending from the proximal end (14, 28) to the distal end (16, 30), wherein said distal ends (16, 30) have an area greater than an area of said proximal ends (14, 28), said first and second bone dowels (12, 26) comprising:
i. particulate bone of between 75 and 600 microns, powdered bone of 75 microns or smaller in size, or both, and
ii. a calcium sulfate hemihydrate, a calcium phosphate product, or both;
(b) providing a ligament (20) having a first end (22) and a second end (24);
(c) drilling a first tunnel through a first bone of the patient;
(d) drilling a second tunnel through a second bone of the patient;
(e) threading the ligament (20) through the first tunnel and second tunnel in any order;
(f) threading and attaching the second end (24) of the ligament (20) through the second proximal end (28) of said second bone dowel (26) and attaching said second end (24) of the ligament (20) within said second axial bore (32) such that the proximal ends (14, 28) of the first and second bone dowel (12, 26) are closer to each other than the distal ends (16, 30) of the first and second bone dowel (12, 26) are to each other thereby securing said ligament (20) to said first bone and said second bone and producing a secured bone connection.

19. The method of claim 18, wherein said first bone dowel (12) and said ligament (20) are provided as a composite graft (10), wherein said first end (22) of said of said ligament (20) is attached to said first bone dowel (12) such that the proximal end (14) of said first bone dowel (12) is closer to the second end (24) of said ligament (20) than the distal end (16) of said first bone dowel (12).

20. The method of claim 18 wherein said distal end (16) of said first bone dowel (12), said distal end (30) of said second bone dowel (26), or both (16, 30) have an area greater than or equal to a cross-sectional area of said first tunnel, said second tunnel, or both, respectively.

21. The method of claim 18 wherein said first tunnel, said second tunnel or both comprise a wide end and a narrow end, said wide end adapted for receiving and securing said first or second bone dowel (12, 26) and to prevent said first or second bone dowel (12, 26) from traversing through said first or second tunnel.

22. The method of claim 18, wherein said damaged ligament in the patient is an anterior cruciate ligament, said first bone is a tibia, and said second bone is a femur.

23. The method of claim 18 wherein said secured bone joint does not contain any metal or bone screws.

24. A solid implantable bone construct (12) comprising:
(a) a bone component comprising bone particulate of between 75 and 600 microns, powdered bone of 75 microns or smaller in size, or both, wherein said solid implantable bone construct is a shape selected from a cylinder, a cone, or a frustum; and
(b) a biocompatible solid component comprising calcium sulfate hemihydrate, a calcium phosphate product, or both.

25. The solid implantable bone construct (12) of claim 24 wherein said bone component comprises between 5 and 50 wt-% of said implant and said biocompatible solid component comprises at least 50 wt-% of said implant.

26. The solid implantable bone construct (12) of claim 24 wherein said bone component comprises between 5 and 30 wt-% of said implant and said biocompatible solid component comprises at least 70 wt-% of said implant.

27. The solid implantable bone construct (12) of claim 24 wherein said particulate bone is freeze dried bone, frozen bone or unfrozen bone.

28. The solid implantable bone construct (12) of claim 24 wherein said particulate bone is a mixture of sizes between 75 and 600 microns.

29. The solid implantable bone construct (12) of claim 24, wherein said solid implantable construct comprises an axial bore (18).

30. The solid implantable bone construct (12) of claim 29 wherein said calcium phosphate product is selected from the group consisting of calcium deficient apatite, hydroxyapatite, beta-tricalcium phosphate, biphasic calcium phosphate, and a combination thereof.

31. A bone repair composition comprising
(a) a bone component comprising bone particulate of between 75 and 600 microns, powdered bone of 75 microns or smaller in size, or both, wherein said solid implantable bone construct is a shape selected from a cylinder, a cone, or a frustum; and
(b) a biocompatible solid component comprising calcium sulfate hemihydrate, a calcium phosphate product, or both,
wherein said bone component comprises between 5 and 50 wt-% and said biocompatible solid component comprises at least 50 wt-%, both based on a total weight of said bone component and said biocompatible solid component.

32. The bone repair composition according to claim 31, wherein said bone component comprises between 5 and 30 wt-% and said biocompatible solid component comprises at least 70 wt-% both based on said total weight of said bone component and said biocompatible solid component.

33. The bone repair composition according to claim 31, further comprising water, wherein water is present in an amount between 0.10 ml and 0.32 ml per gram of said total weight of said bone component and said biocompatible solid component.
